(19) United States
(12) Patent Application Publication
(10) Pub. No.: US 2004/0093087 A1
(43) Pub. Date: May 13, 2004

(54) Fluid-filled artificial disc replacement (ADR)

(76) Inventors: Bret A. Ferree, Cincinnati, OH (US); David Tompkins, Milford, OH (US)

Correspondence Address: John G. Posa, Gifford, Krass, Groh, Sprinkle,, Anderson & Citkowski, P.C., 280 N. Old Woodward Ave., Suite 400, Birmingham, MI, 48009-5394, US

(21) Appl. No.: 10/701,893

(22) Filed: Nov. 5, 2003

Related U.S. Application Data
(60) Provisional application No. 60/423,885, filed on Nov. 5, 2002.
(60) Provisional application No. 60/434,894, filed on Dec. 19, 2002.

(51) Int. Cl. A61F 2/44; A61F 2/30

(52) U.S. Cl. 623/17.13; 623/23.41

(57) ABSTRACT

Fluids and/or elastomeric materials are used to dampen forces across rigid endplates in an artificial disc replacement (ADR) or other artificial joints within the body (animal or human) including, for example, the tibial component of a knee replacement. Preferred embodiments use a fluid/elastomer combination to dampen forces in the ADR. Much like the normal human disc, fluid within the center of the ADR transfers compressive loads to a component surrounding the fluid. The surrounding component, preferably an elastomer, expands to dampen the forces transmitted by the fluid. According to the invention, a flat elastomeric ring is positioned adjacent to a flat inner surface of the ADR endplates. Alternatively, the invention may also use a convex shaped elastomer ring adjacent to concave inner surfaces of the ADR endplates; a concave shaped elastomer ring adjacent to convex inner surfaces of the ADR endplates; a convex surface on one side of the elastomer ring and a flat surface on the other side of the elastomer ring; or any combination of surface shapes on the elastomer ring and the inner surface of the ADR endplates. Hydrogels may be used within the elastomer ring and enclosed, for example, within a porous bag. Alternatively, free hydrogel material may be placed within the elastomeric ring without a container. In such instances, the elastomeric ring or the ADR endplates or both would preferably contain pores for the movement of fluids into and out of the hydrogel. Optionally, spikes or other projections may be used to assist in fixing the endplates to respective vertebral bodies or articulating bones.

### REFERENCE TO RELATED APPLICATIONS

This application claims priority from U.S. Provisional Patent Application Serial No. 60/423,885, filed Nov. 5, 2002 and 60/434,894, filed Dec. 19, 2002; the entire content of each application being incorporated herein by reference.

### FIELD OF THE INVENTION

This invention relates generally to artificial disc replacements (ADRs) and, in particular, to the use of fluids and/or elastomeric materials to dampen forces across rigid endplates in an ADR.

### BACKGROUND OF THE INVENTION

Eighty-five percent of the population will experience low back pain at some point. Fortunately, the majority of people recover from their back pain with a combination of benign neglect, rest, exercise, medication, physical therapy, or chiropractic care. A small percent of the population will suffer chronic low back pain. The cost of treatment of patients with spinal disorders plus the patient's lost productivity is estimated at 25 to 100 billion dollars annually.

Seven cervical (neck), 12 thoracic, and 5 lumbar (low back) vertebrae form the normal human spine. Intervertebral discs reside between adjacent vertebra with two exceptions. First, the articulation between the first two cervical vertebrae does not contain a disc. Second, a disc lies between the last lumbar vertebra and the sacrum (a portion of the pelvis).

The spine supports the body, and protects the spinal cord and nerves. The vertebrae of the spine are also supported by ligaments, tendons, and muscles which allow movement (flexion, extension, lateral bending, and rotation). Motion between vertebrae occurs through the disc and two facet joints. The disc lies in the front or anterior portion of the spine. The facet joints lie laterally on either side of the posterior portion of the spine.

The human intervertebral disc is an oval to kidney bean shaped structure of variable size depending on the location in the spine. The outer portion of the disc is known as the annulus fibrosis. The annulus is formed of 10 to 60 fibrous bands. The fibers in the bands alternate their direction of orientation by 30 degrees between each band. The orientation serves to control vertebral motion (one half of the bands tighten to check motion when the vertebra above or below the disc are turned in either direction). The annulus contains the nucleus. The nucleus pulpous serves to transmit and dampen axial loads. A high water content (70-80 percent) assists the nucleus in this function. The water content has a diurnal variation. The nucleus imbibes water while a person lies recumbent. Activity squeezes fluid from the disc. Nuclear material removed from the body and placed into water will imbibe water swelling to several times its normal size. The nucleus comprises roughly 50 percent of the entire disc. The nucleus contains cells (chondrocytes and fibrocytes) and proteoglycans (chondroitin sulfate and keratin sulfate). The cell density in the nucleus is on the order of 4,000 cells per micro liter.

Interestingly, the adult disc is the largest avascular structure in the human body. Given the lack of vascularity, the nucleus is not exposed to the body's immune system. Most cells in the nucleus obtain their nutrition and fluid exchange through diffusion from small blood vessels in adjacent vertebra.

The disc changes with aging. As a person ages the water content of the disc falls from approximately 85 percent at birth to 70 percent in the elderly. The ratio of chondroitin sulfate to keratin sulfate decreases with age. The ratio of chondroitin 6 sulfate to chondroitin 4 sulfate increases with age. The distinction between the annulus and the nucleus decreases with age. These changes are known as disc degeneration. Generally disc degeneration is painless.

Premature or accelerated disc degeneration is known as degenerative disc disease. A large portion of patients suffering from chronic low back pain are thought to have this condition. As the disc degenerates, the nucleus and annulus functions are compromised.

The nucleus becomes thinner and less able to handle compression loads. The annulus fibers become redundant as the nucleus shrinks. The redundant annular fibers are less effective in controlling vertebral motion. The disc pathology can result in: 1) bulging of the annulus into the spinal cord or nerves; 2) narrowing of the space between the vertebra where the nerves exit; 3) tears of the annulus as abnormal loads are transmitted to the annulus and the annulus is subjected to excessive motion between vertebra; and 4) disc herniation or extrusion of the nucleus through complete annular tears.

Current surgical treatments of disc degeneration are destructive. One group of procedures removes the nucleus or a portion of the nucleus; lumbar discectomy falls in this category. A second group of procedures destroy nuclear material; Chymopapin (an enzyme) injection, laser discectomy, and thermal therapy (heat treatment to denature proteins) fall in this category. A third group, spinal fusion procedures either remove the disc or the disc's function by connecting two or more vertebra together with bone. These destructive procedures lead to acceleration of disc degeneration. The first two groups of procedures compromise the treated disc. Fusion procedures transmit additional stress to the adjacent discs. The additional stress results in premature disc degeneration of the adjacent discs.

Prosthetic disc replacement offers many advantages. The prosthetic disc attempts to eliminate a patient's pain while preserving the disc's function. Current prosthetic disc implants, however, either replace the nucleus or the nucleus and the annulus. Both types of current procedures remove the degenerated disc component to allow room for the prosthetic component. Although the use of resilient materials has been proposed, the need remains for further improvements in the way in which prosthetic components are incorporated into the disc space, and in materials to ensure strength and longevity. Such improvements are necessary, since the prosthesis may be subjected to 100,000,000 compression cycles over the life of the implant.

### SUMMARY OF THE INVENTION

Broadly, this invention utilizes fluids and/or elastomeric materials to dampen forces across rigid endplates in an artificial disc replacement (ADR). The invention can also be used to dampen other artificial joints within the body (animal or human) including, for example, the tibial component of a knee replacement.

Preferred embodiments use a fluid/elastomer combination to dampen forces in the ADR. Much like the normal human disc, fluid within the center of the ADR transfers compressive loads to a component surrounding the fluid. The surrounding component, preferably an elastomer, expands to dampen the forces transmitted by the fluid.

According to the invention, a flat elastomeric ring is positioned adjacent to a flat inner surface of the ADR endplates. Alternatively, the invention may also use a convex shaped elastomer ring adjacent to concave inner surfaces of the ADR endplates; a concave shaped elastomer ring adjacent to convex inner surfaces of the ADR endplates; a convex surface on one side of the elastomer ring and a flat surface on the other side of the elastomer ring; or any combination of surface shapes on the elastomer ring and the inner surface of the ADR endplates.

Hydrogels may be used within the elastomer ring and enclosed, for example, within a porous bag. Alternatively, free hydrogel material may be placed within the elastomeric ring without a container. In either embodiment, the elastomeric ring or the ADR endplates or both would preferably contain pores for the movement of fluids into and out of the hydrogel.

Optionally, spikes or other projections may be used to assist in fixing the endplates to respective vertebral bodies. The spikes or the projections from the ADR endplates may also vary in height. For example, the spikes closest to the anterior portion of the ADR may be longer than the spikes at the posterior portion of the ADR. Alternatively, the spikes on the anterior portion of the ADR endplates may be shorter than the spikes on the posterior portion of the ADR.

The ADR endplates may additionally be differing in thickness. For example, the upper ADR endplate could be thicker than the lower endplate. The convexity of the bone surface of the ADR does not need to be centered. Alternatively, the thickest portion of the ADR endplate may be posterior to the midline.

A flexible impermeable membrane could be sealed to the ADR endplates. The membrane would protect the elastomer component from exposure to the body fluids and cells. Furthermore, the membrane could protect the body form elastomer wear debris.

The description also discloses the use of a component that attaches to one or both ADR endplates to prevent the extrusion of a component or components from between the ADR endplates. The anti-extrusion component can be added to the ADR endplate after insertion of the other component or components between the ADR endplates. Components attached to the ADR endplate or endplates may change shape, size or position to prevent the extrusion of a component or components from the space between the ADR endplates.

### BRIEF DESCRIPTION OF THE DRAWINGS

FIG. 1 is a view of the lateral aspect of an ADR constructed according to the invention;

FIG. 2 is a view of the anterior aspect of the ADR of FIG. 1;

FIG. 3A is a sagittal cross section of the ADR of FIG. 1;

FIG. 3B is an alternative embodiment of the ADR drawn in FIG. 3A;

FIG. 4 is an exploded view;

FIG. 5A is a sagittal cross section of another embodiment of an ADR according to the invention;

FIG. 5B is a sagittal cross section of an alternative embodiment of the ADR of FIG. 5A;

FIG. 6 is a sagittal cross section of an ADR with a pressure-limiting feature;

FIG. 7A is a view of the lateral aspect of the spine and a distraction guide;

FIG. 7B is a is a view of the lateral aspect of the spine, a guide, and the shaver;

FIG. 7C is a sagittal cross section of the guide and the shaver;

FIG. 7D is a view of the side of the shaver;

FIG. 7E is a view of the side of the shaver rotated 90 degrees from the orientation drawn in FIG. 7D;

FIG. 7F is a view of the end of a guide;

FIG. 8A is a cross section through another embodiment of an ADR constructed in accordance with the invention;

FIG. 8B is a view of the top of the elastomer ring and plates drawn in FIG. 8A;

FIG. 9 is a cross section of an alternative embodiment of an ADR wherein elastomeric cushions are placed between the fluid filled bag and the metal endplates;

FIG. 10 is a top view of an ADR guide according to the invention that assists a surgeon in determining the proper size of the ADR to use;

FIG. 11A is a view of an instrument used to form pilot holes;

FIG. 11B is a view of an alternative embodiment of the instrument drawn in FIG. 11A;

FIG. 12 is a view of the top or bottom of an ADR endplate;

FIG. 13 is a view of the lateral surface of an ADR;

FIG. 14A is a view of the side of the ADR with movable projections from the ADR endplates that serve to prevent the extrusion of the removable cushioning element of the ADR;

FIG. 14B is a view of the embodiment of the ADR drawn in FIG. 14A with the projections in a retracted position;

FIG. 14C is a sagittal cross section of the embodiment of the ADR drawn in FIGS. 14A and 14B;

FIG. 15A is an enlarged sagittal cross section of an alternative embodiment of the retractable projections drawn in FIGS. 14A, 14B and 14C;

FIG. 15B is view of extended retractable projections drawn in FIG. 15A;

FIG. 16A is an exploded view of the side of an alternative embodiment of the ADR with an attachable piece used to prevent the extrusion of the cushioning element;

FIG. 16B is an enlarged view of the embodiment of the ADR drawn in FIG. 16A with an anti-extrusion piece attached;

FIG. 16C is a view of the front of the ADR drawn in FIGS. 16A and 16B;

FIG. 17A is the view of the side of an alternative embodiment of an ADR with features to prevent the extrusion of the cushioning component;

FIG. 17B is an enlarged view of the embodiment of the device to prevent extrusion of the cushioning device drawn in FIG. 17A;

FIG. 17C is a view of the front of the embodiment of the ADR drawn in FIGS. 17A and 17B;

FIG. 18 is side view of an alternative embodiment of an ADR according the invention FIG. 19A is the view of the side of an alternative embodiment of the ADR with a component attached to the front of the ADR endplate to prevent extrusion of the elastomer ring;

FIG. 19B is a view of the component that snaps to the ADR endplate to prevent the extrusion of the elastomer ring;

FIG. 20 is a sagittal cross-section of an embodiment of the ADR with asymmetric endplates;

FIG. 21 is a sagittal cross section of an alternative embodiment wherein the ADR endplates and elastomer cooperate to hold a hydrogel or fluid-filled bag in position;

FIG. 22 is a sagittal cross section of an alternative embodiment of an ADR with endplates similar to those drawn in FIG. 21;

FIG. 23 is a cross section of yet a further alternative embodiment of an ADR according to this invention including an elastomer or a fiber material such as Gortex;

FIG. 24A is a view of the side of another embodiment of the ADR including an elastomeric ring;

FIG. 24B is a view of the side of the ADR drawn in FIG. 24A and the spine;

FIG. 24C is a view of the vertebral side of the ADR endplate drawn in FIG. 24A;

FIG. 25A is a sagittal cross section of the ADR drawn in FIG. 24A and the spine;

FIG. 25B is a sagittal cross section of the modular cushioning component drawn in FIG. 20A;

FIG. 25C is a sagittal cross section of the ADR endplates with the spring-loaded projections;

FIG. 26A is a view of the top of a guide for creating grooves in the vertebrae for the fins or supports of the ADR endplates shown in FIG. 24A;

FIG. 26B is the side view of an example of a 90-degree bur or drill 2110 much like those used in dentistry;

FIG. 26C is a view of the top of a second guide used to create groves in the second vertebra;

FIG. 26D is a view of the side of a portion of the guide drawn in FIG. 26C;

FIG. 26E is a view of the top of a third guide used to complete the grooves in the second vertebra;

FIG. 26F is a view of the side of a portion of the guide drawn in FIG. 26E;

FIG. 27A is a sagittal cross section of another embodiment of the ADR endplates with an alternative locking mechanism;

FIG. 27B is a sagittal cross section of the modular cushioning component that fits into the embodiment of the ADR endplates drawn in FIG. 27A;

FIG. 27C is a sagittal cross section of an assembled embodiment of the ADR drawn in FIGS. 27A and 27B; and

FIG. 28 is the view of the side of an alternative embodiment of the ADR. Physical features associated with the top and bottom endplates interact to limit the amount of torsional rotation allowed by the ADR.

### DETAILED DESCRIPTION OF THE INVENTION

Broadly, this invention uses a fluid/elastomer combination to dampen forces across metal endplates in an artificial disc replacement (ADR). However, the invention can also be used to dampen other artificial joints within the body (animal or human) including, for example, the tibial component of a knee replacement.

Much like the normal human disc, fluid within the center of the ADR transfers compressive loads to a component surrounding the fluid. The surrounding component, preferably an elastomeric, expands to dampen the forces transmitted by the fluid.

The fluid within the center of the ADR may be contained in a separate elastomeric bag, free within the center of the elastomer ring, or contained in hydrogels within the center of the elastomer ring. The hydrogel-containing embodiments may accommodate a water permeable elastomer or water permeable endplate component. The metal endplates preferably have a bone growth surface on one side, and a highly polished surface on the other that cooperates with the elastomeric components. A metal plate may be bonded to the top and bottom of the outer elastomer ring to further reduce the friction between the elastomer ring and the metal endplates. Alternatively, the metal plate could contain a projection to fit within a groove within the elastomeric component to loosely attach the metal and elastomeric components.

The preferred embodiment uses saline, biocompatible oils, or other fluids within the ADR. Alternatively, the bag within the center of the device could be filled with air, gas, gels (including hydrogels), or polymers.

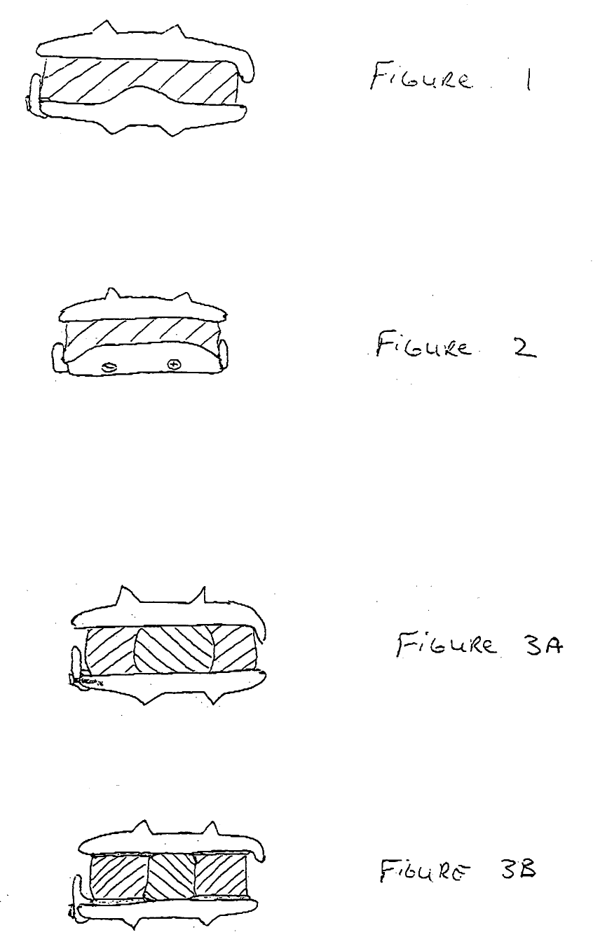

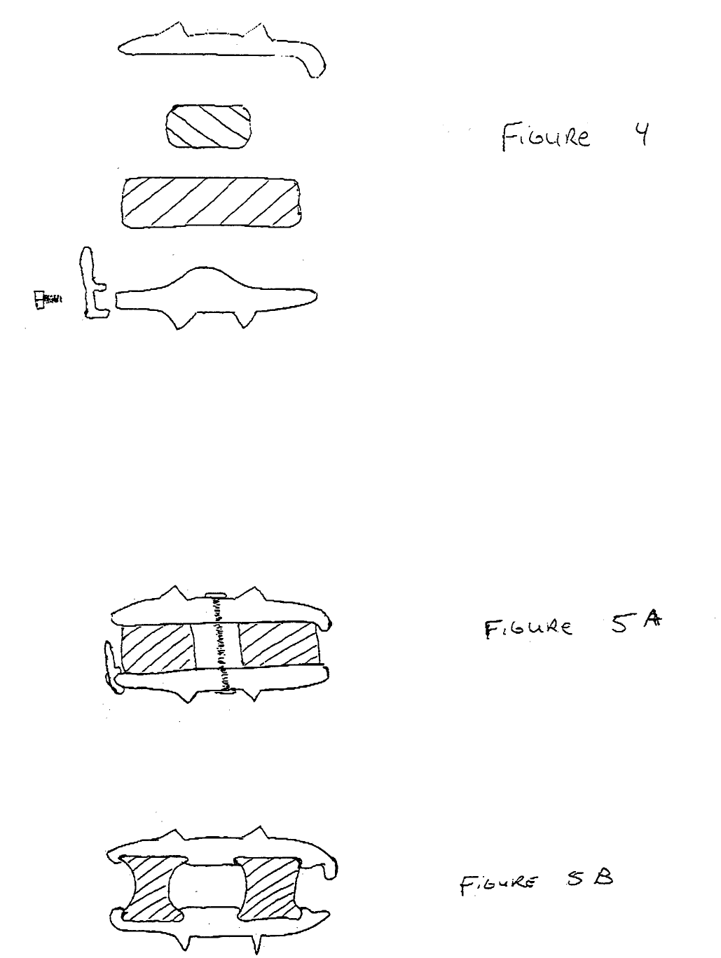

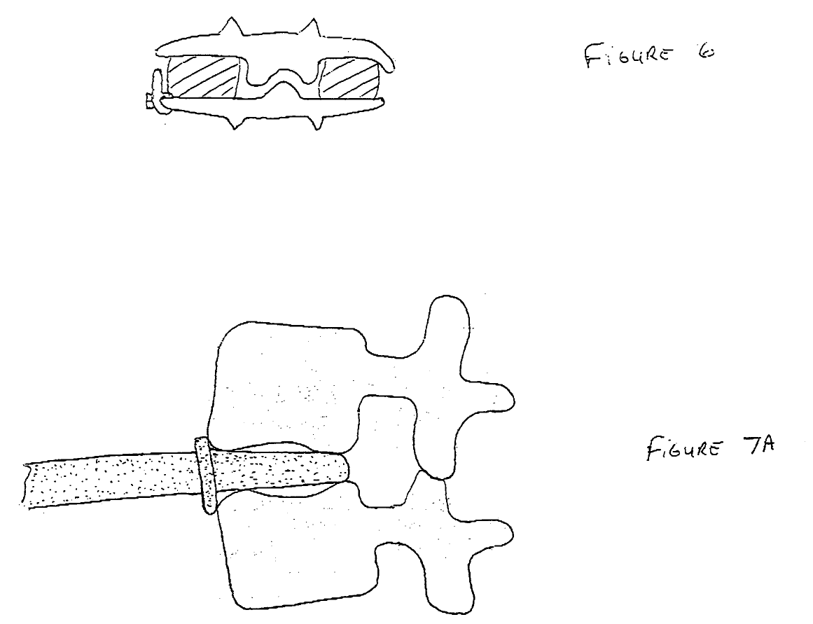

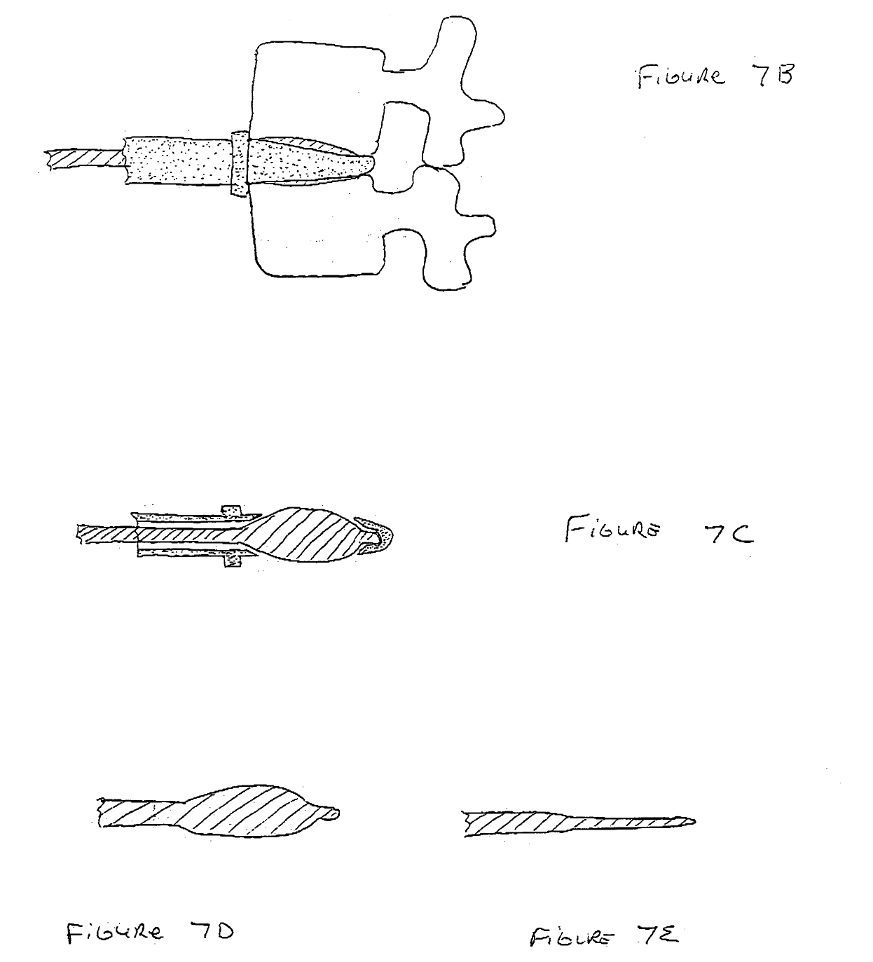

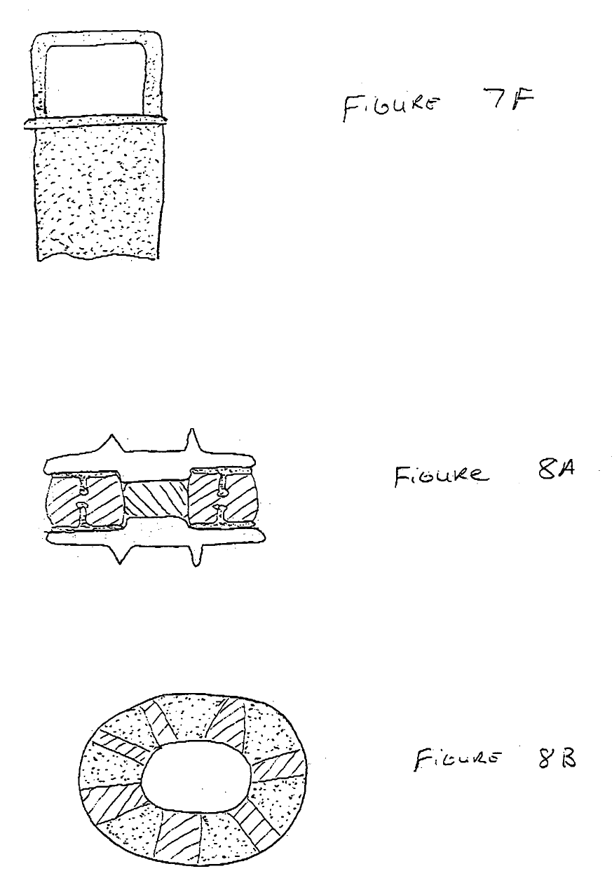

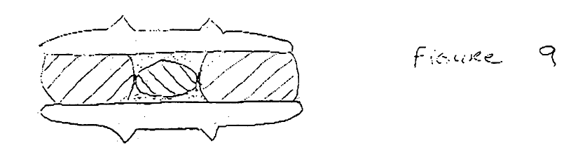

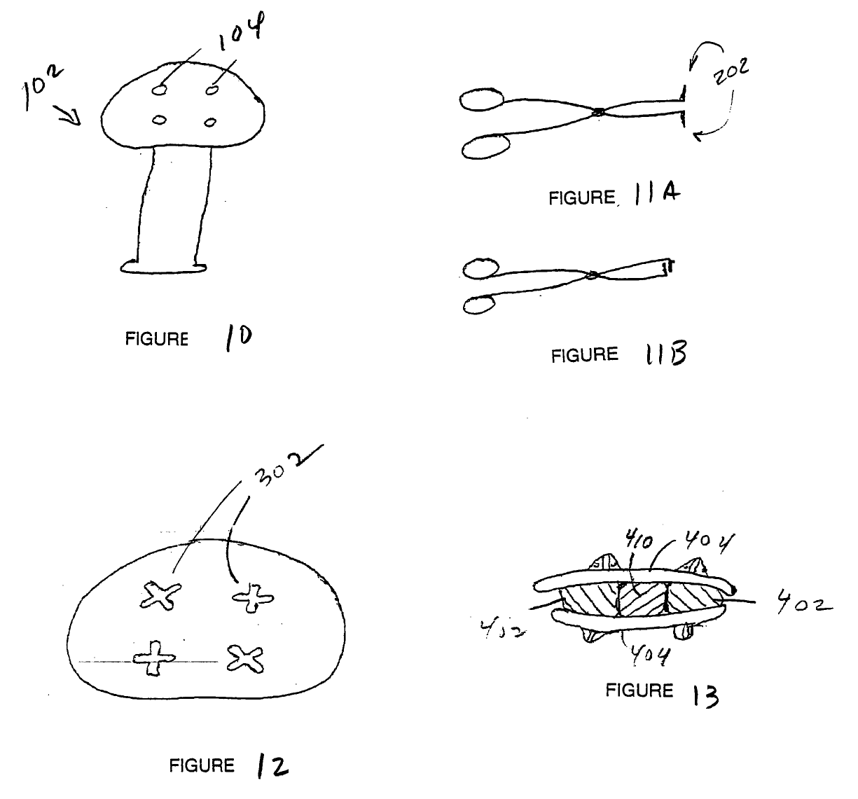

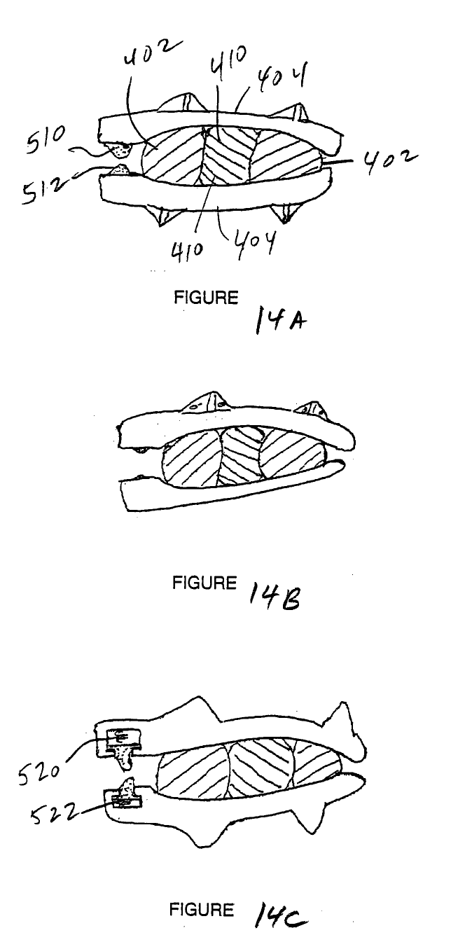

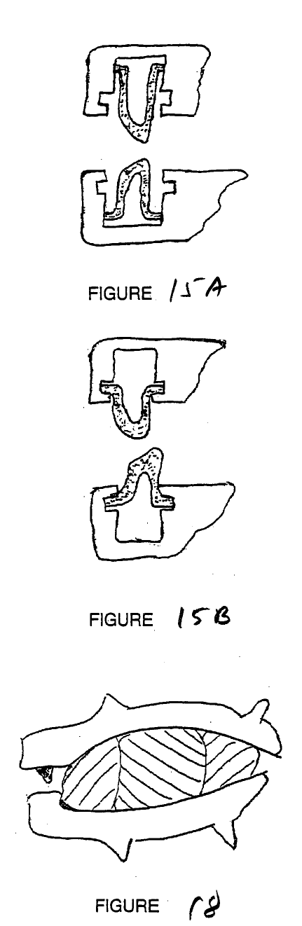

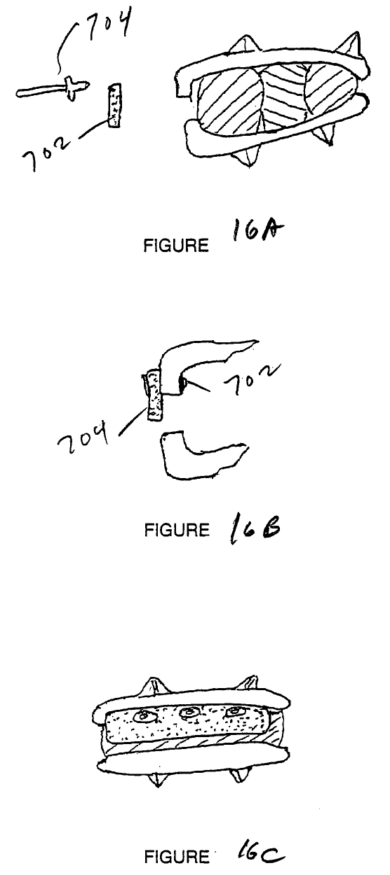

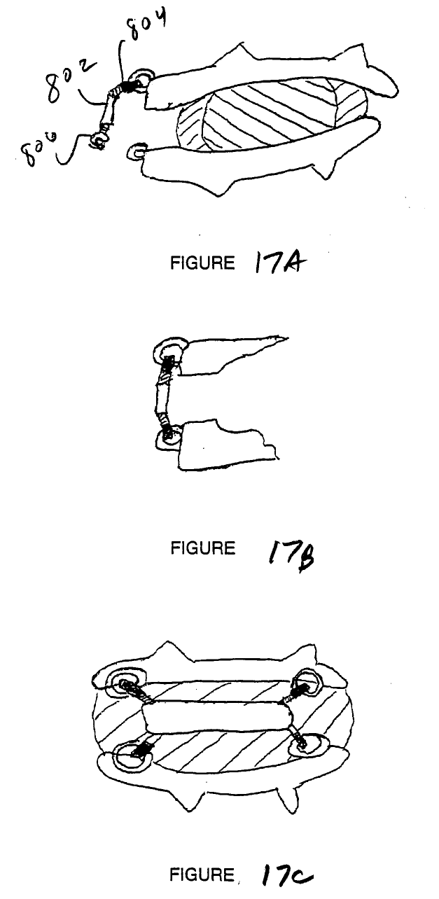

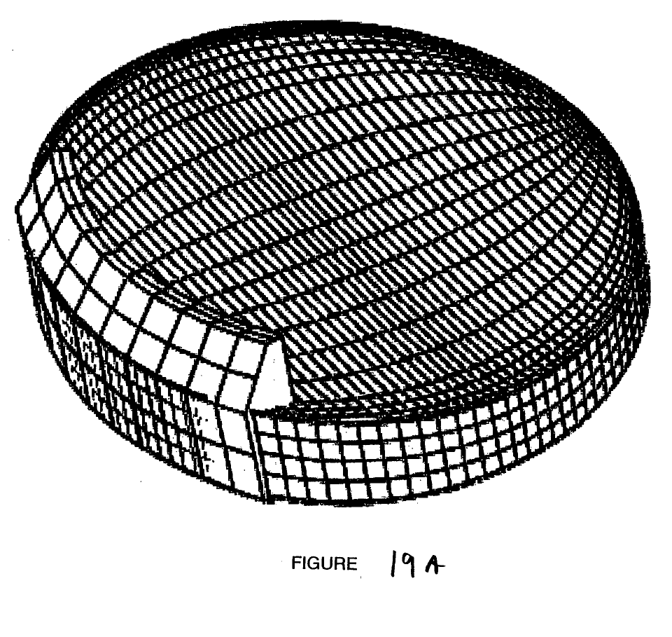

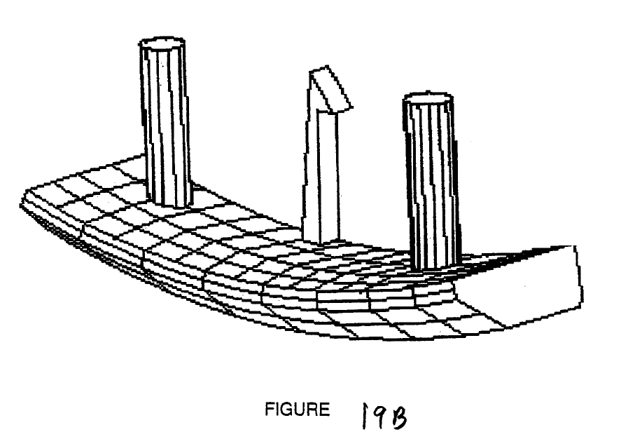

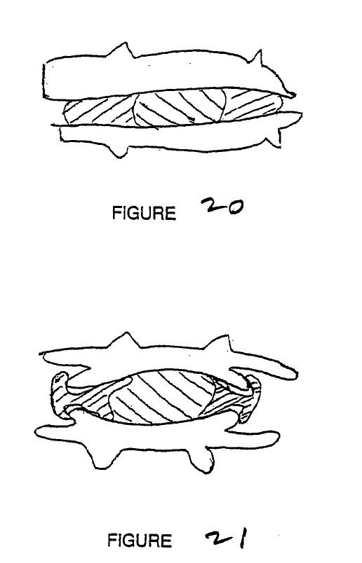

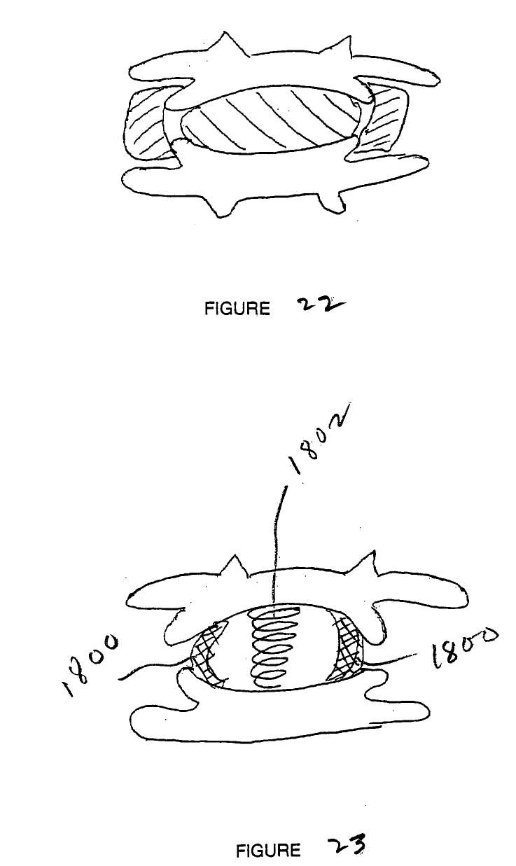

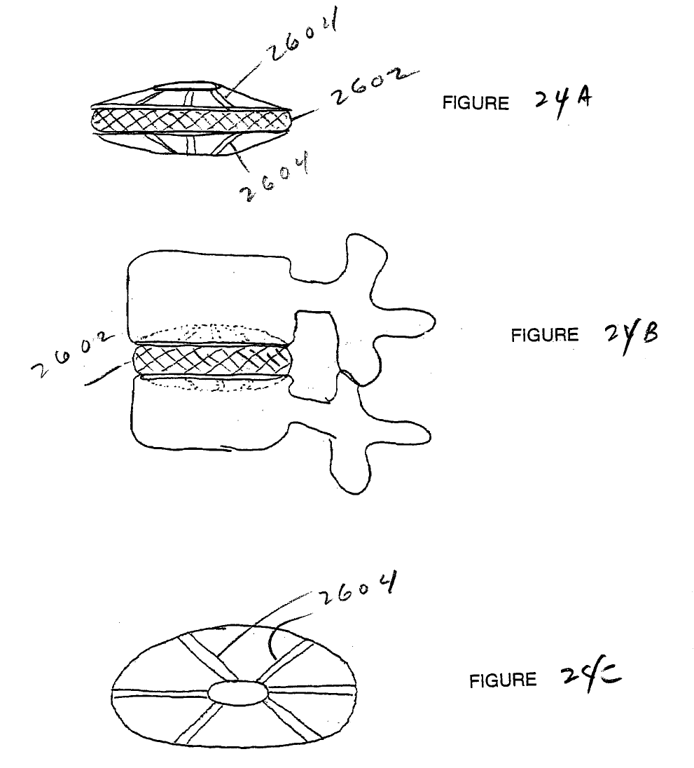

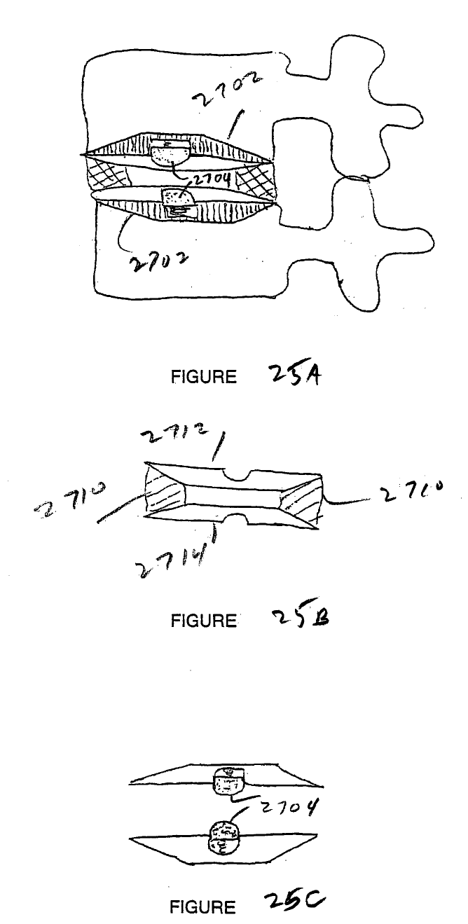

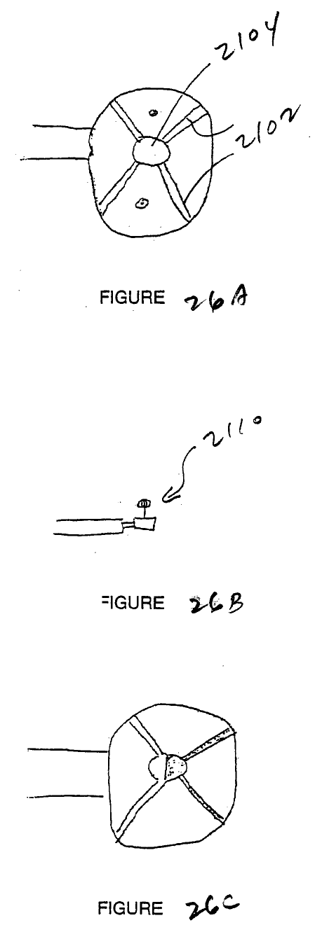

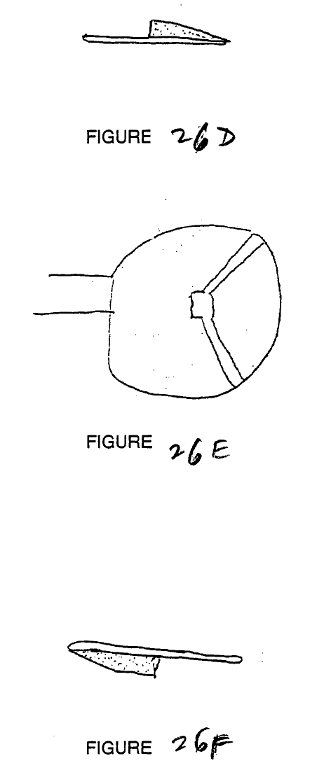

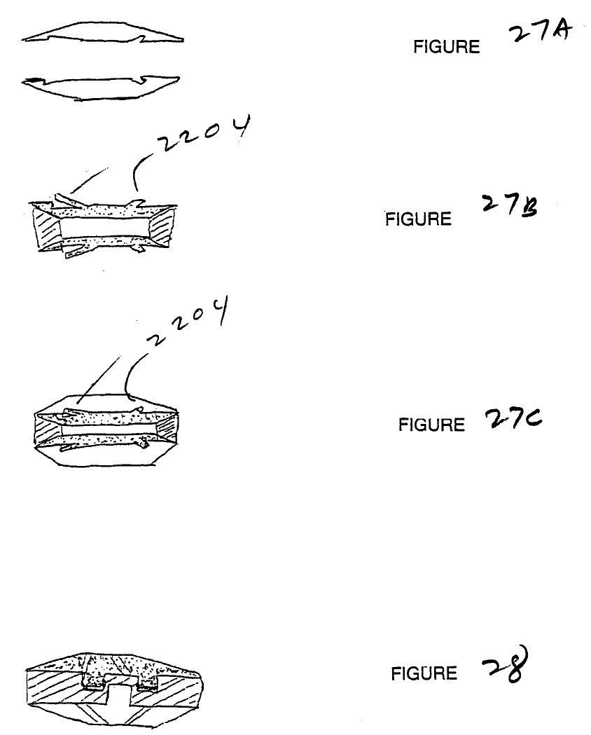

FIG. 1 is a view of the lateral aspect of an ADR constructed according to the invention. FIG. 2 is a view of the anterior aspect of the ADR. The cross-hatched area represents ring shaped elastomer. The metal endplates have spikes to hold the ADR between the vertebrae above and below the ADR. The sides of the endplates have projections to hold the elastomeric component between the endplates. A plate can be added to the anterior aspect of the ADR to hold the elastomeric component in position after the elastomeric component is placed between the ADR endplates. The plate could be laser welded at the time of surgery.

FIG. 3A is a sagittal cross section of the ADR. The central area contains a fluid, such as saline. The fluid can be contained with an elastomeric bag or a hydrogel. The hydrogel could be free within the space within the elastomer. Alternatively, the hydrogel could be placed in a fluid permeable bag within the space within the elastomer. An expandable membrane or material could be placed between the elastomer and the fluid filled bag to reduce friction between the elastomer and bag.

FIG. 3B is an alternative embodiment of the ADR drawn in FIG. 3A. Thin metal, plastic, polymer, or polyethylene plates are attached to the top and bottom of the elastomeric ring (dotted area of the drawing). FIG. 4 is an exploded view of the ADR. The plate on the anterior aspect of the inferior ADR endplate can be attached to the endplate with screws, clips, or by other mechanisms.

FIG. 5A is a sagittal cross section of another embodiment of the ADR. Fluid is held freely within the ADR by the fit between the elastomer ring and the ADR endplates. When positioned with the body, pressure from the superior and inferior endplates will help seal the interface between the ADR endplates and the elastomeric ring. An optional cable could be used to maintain compression of the elastomer.

FIG. 5B is a sagittal cross section of an alternative embodiment of the ADR drawn in FIG. 5A. The ADR endplates may have features that cooperate with the elastomer ring to improve the seal between the elastomer and the endplates.

FIG. 6 is a sagittal cross section of the ADR with a pressure-limiting feature. Metal projections from the endplates impinge once the ADR is subjected to more than a certain force. The metal projections could allow unrestricted motion between the endplates until the pressure on the ADR is high enough to force the metal projections together. Alternatively, the metal projections could restrict certain motion, for example translation, before enough axial load is applied to the ADR to force the projections together tightly. In either case, when the load on the ADR exceeds a certain amount, for example 350 P.S.I. the metal projections carry the additional load. The metal projections protect the elastomeric ring from excessive pressure.

FIG. 7A is a view of the lateral aspect of the spine and a distraction guide. The guide distracts the vertebrae to restore normal disc height. The guide also cooperates with a twist shaver to contour the endplates of the vertebrae. The shaved endplates increase the surface contact between the ADR endplates and the vertebral endplates. The improved surface contact improves bone ingrowth into the ADR endplates.

FIG. 7B is a view of the lateral aspect of the spine, the guide (dotted area), and the shaver (cross hatched area). The convex sides of the shaver create concave cavities within the vertebral endplates. The dense cortical bone surrounding the periphery of the vertebral endplates is preserved to support the ADR.

FIG. 7C is a sagittal cross section of the guide (dotted area) and the shaver (cross hatched area). FIG. 7D is a view of the side of the shaver. The guide cooperates with the end of the shaver and the shaft of the shaver to enable the surgeon to precisely shape the vertebrae.

FIG. 7E is a view of the side of the shaver rotated 90 degrees from the orientation drawn in FIG. 7D. The flat shape of the shaver enables it to be inserted into the guide and the disc space through a narrow opening. Once inside the disc space, the shave is rotated to cut the vertebrae.

FIG. 7F is a view of the end of the guide. The guide has an opening for the end of the shaver. Different sized shavers could be used to create a biconcave space for a biconvex ADR. Alternatively, a single shaver could be used to create a cylinder shaped space for a cylinder shaped ADR.

FIG. 8A is a cross section through another embodiment of the ADR. Reduced friction plates are used on the top and bottom of the elastomeric ring (dotted area of the drawing). The reduced friction plates are shaped to fit with the elastomer ring such that the plates are not glued to the elastomer ring. The metal endplates have protrusions to help hold the fluid filled bag and elastomer ring in position. The projections from the periphery of the endplates can be eliminated in this embodiment.

FIG. 8B is a view of the top of the elastomer ring and plates drawn in FIG. 8A. FIG. 9 is a cross section of an alternative embodiment of the ADR, wherein elastomer cushions are placed between the fluid filled bag and the metal endplates.

FIG. 10 is a top view of an ADR guide according to the invention that assists a surgeon in determining the proper size of the ADR to use. Holes 104 within the guide 102 also allow the surgeon to make pilot holes for the spikes or projections on the ADR endplates. FIG. 11A is a view of an instrument used to form the pilot holes. Sharp points 202 on the instrument are forced through the alignment holes of the ADR guide. The instrument can also be rotated within the holes of the guide to effectively “drill” the pilot holes in the vertebrae. FIG. 11B is a view of an alternative embodiment of the instrument drawn in FIG. 11A. Yet another embodiment of the guide and hole drilling instrument could be made by combining the instruments. For example, the ADR guide could have spikes that could be used to form the pilot holes.

FIG. 12 is a view of the top or bottom of the ADR endplate. Projections 302 are preferably oriented in more than one direction to improve the attachment of the ADR endplate to the vertebrae. FIG. 13 is a view of the lateral surface of the ADR. A convex elastomer ring 402 and with ADR endplates 404 with concave inner surfaces is shown in this embodiment. A fluid-filled bag 410 is contained by the elastomer ring and the ADR endplates.

FIG. 14A is a view of the side of the ADR with movable projections 510, 512 from the ADR endplates that serve to prevent the extrusion of the removable cushioning element of the ADR. The projections 510, 512 retract during insertion of the cushioning element. The projections also retract if they impinge against one another during spinal movement.

FIG. 14B is a view of the embodiment of the ADR drawn in FIG. 14A with the projections in the retracted position. FIG. 14C is a sagittal cross section of the embodiment of the ADR drawn in FIGS. 14A and 14B. Springs 520, 522 may be used to encourage the projections to close the opening in the front of the ADR. The retractable projections can be located anywhere around the periphery of the ADR cushioning element.

FIG. 15A is an enlarged sagittal cross section of an alternative embodiment of the retractable projections drawn in FIGS. 14A, 14B and 14C. A spring biased retractable projection is preferably used. FIG. 15B is view of extended retractable projections drawn in FIG. 15A. Unlike the retractable projections drawn in FIGS. 14A, 14B and 14C, the projections in this embodiment can be locked in the extended position.

FIG. 16A is an exploded view of the side of an alternative embodiment of the ADR with an attachable piece 702 to prevent the extrusion of the cushioning element. The attachable piece can be attached with a fastener such as one or more pop rivets 704. FIG. 16B is an enlarged view of the embodiment of the ADR drawn in FIG. 16A with the anti-extrusion piece (dotted area of the drawing) attached. FIG. 16C is a view of the front of the ADR drawn in FIGS. 16A and 16B.

FIG. 17A is the view of the side of an alternative embodiment of an ADR with features to prevent the extrusion of the cushioning component. A band 802 is attached to both ADR endplates with cables 804, 806. The cables allow movement of one ADR endplate relative to another. The inferior cables are attached to the inferior ADR endplate after insertion of the cushioning element. The cables can extend through holes in the band used to prevent extrusion of the cushioning device. FIG. 17A also illustrates the variability of the ratio of the fluid filled bag to the elastomeric ring. In this illustration, the fluid filled bag occupies a larger area than the elastomer ring. FIG. 17B is an enlarged view of the embodiment of the device to prevent extrusion of the cushioning device drawn in FIG. 17A. FIG. 17C is a view of the front of the embodiment of the ADR drawn in FIGS. 17A and 17B.

FIG. 18 is side view of an alternative embodiment of an ADR according the invention. A raised portion of the ADR endplate from one side can cooperate with a movable or attachable mechanism form the second ADR endplate to hold the cushioning element in position. FIGS. 17A and 18 also illustrate that the cushioning element may sit somewhat posterior to the midline of the ADR endplates.

FIG. 19A is the view of the side of an alternative embodiment of the ADR with a component attached to the front of the ADR endplate to prevent extrusion of the elastomer ring. FIG. 19B is a view of the component that snaps to the ADR endplate to prevent the extrusion of the elastomer ring.

FIG. 202 is a sagittal cross-section of an embodiment of the ADR with asymmetric endplates. The upper endplate is thicker than the lower endplate. The maximum thickness of the endplates is posterior to the midline. FIG. 21 is a sagittal cross section of an alternative embodiment wherein the ADR endplates and elastomer cooperate to hold a hydrogel or fluid-filled bag in position.

FIG. 22 is a sagittal cross section of an alternative embodiment of the ADR with endplates similar to those drawn in FIG. 21. The elastomer ring is lateral to the raised portions of the ADR endplates in this embodiment.

FIG. 23 is a cross section of yet a further alternative embodiment of an ADR according to this invention including an elastomer 1800 or a fiber material such as Gortex. A spring 1802 is added to the space for fluid or hydrogel. As a final note, although a fluid-filled bag is disclosed in the preferred embodiments, a bag filled with air or other gas could instead be used. For example, an air-filled bag surrounded by polyurethane would function similar to an air-filled shoe.

The ADR endplates could have novel structural supports on the bone ingrowth side of the plates. Prior-art ADR endplates are thick and rest upon the vertebral endplates. The disc space is limited, thus the thin cushioning components must be used with thick ADR endplates. The novel ADR endplates place much of the support of the ADR endplate into the vertebrae to increase the amount of space available for a cushioning component.

The structural supports have several important features. First, they extend into the cancellous portion of the vertebrae. Cancellous bone is more likely to grow to the supports than the cortical bone of the endplates. The supports can be covered with a bone growth-promoting surface such as plasma spray. The supports act as fins, increasing the surface area available for bone ingrowth. The supports also resist shear forces between the vertebrae and the endplates, thus facilitating bone ingrowth onto the ADR endplates. The supports resist extrusion of the ADR from the disc space. Furthermore, the ADR endplates cooperate with the modular cushioning component to resist extrusion of the modular cushioning component.

In addition, the plate-like portion of the novel ADR is supported by the strong endplates of the vertebrae. Thus, the ADR is unlikely to “subside” or sink into the soft cancellous bone of the vertebrae. As yet a further advantage, the intra-vertebral location of the supports and the thin plate portion of the novel ADR endplates enable the use of a thicker cushioning component.

FIG. 24A is a view of the side of another embodiment of the ADR including an elastomeric ring 2602. In this case, the ADR endplates have a thin plate-like portion that cooperates with a modular component containing the elastomer ring and a hydrogel. The central portion of the bone ingrowth side of the ADR endplate is raised. The raised central portion is preferably spherical or circular in shape. Reinforcement buttresses or beams 2604 preferably extend from the raised central portion of the endplate to the periphery of the ADR endplate.

FIG. 24B is a view of the side of the ADR drawn in FIG. 24A and the spine. FIG. 24C is a view of the vertebral side of the ADR endplate drawn in FIG. 24A. The raised portion of the endplate extends into the vertebrae as illustrated by the dotted lines. The thin, plate-like portion of the ADR endplate rests against the vertebral endplate. The vertebral endplate can be milled to improve the fit between the ADR endplate and the vertebra.

FIG. 25A is a sagittal cross section of the ADR drawn in FIG. 25A and the spine. A modular cushioning component 2702 is held between the ADR endplates with spring-loaded projections 2704 from the ADR endplates.

FIG. 25B is a sagittal cross section of the modular cushioning component drawn in FIG. 20A. The elastomer ring 2710 is positioned between two pieces of harder material 2712, 2714, which may be metal, plastic, nylon, polyethylene, etc. The space between the polyethylene components and the elastomer ring contains hydrogel or the fluid-filled bag. The polyethylene, elastomer, and/or ADR endplate components may be porous, particularly in the hydrogel embodiment of the device.

FIG. 25C is a sagittal cross section of the ADR endplates with the spring-loaded projections 2704. Polyethylene components would not necessarily require a movable locking mechanism in the ADR endplate. For example, the flexibility of the polyethylene component may enable the use of a locking mechanism similar to those used to lock polyethylene trays into metal tibial components in total knee replacements. A band or clamp could hold the modular cushioning component together until it was placed between the ADR endplates. Note that the polyethylene components can also be inserted separately. The hydrogel and elastomer components can be inserted after the polyethylene components.

FIG. 26A is a view of the top of a guide for creating grooves in the vertebrae for the fins or supports of the ADR endplates shown in FIG. 24A. The handle of the device is not drawn, but would be located on the left side of the drawing. The guide is designed for and ADR endplate with four fins 2102 radiating from a sphere or raised cylinder 2104. The two small circles on either side of the central cylinder represent pins to help hold the guide on the vertebrae.

FIG. 26B is the side view of an example of a 90-degree bur or drill 2110 much like those used in dentistry. The bur is used in the slots of the guide drawn in FIG. 26A, to create the grooves for the fins. The fins or supports of the ADR are wider than the grooves created in the vertebrae to press fit the fins into the vertebrae. FIG. 26C is a view of the top of a second guide used to create groves in the second vertebra. The dotted area of the drawing represents raised areas of the guide that fit into the grooves created in the first vertebra. The second guide helps to align the second ADR endplate with the first ADR endplate. FIG. 26D is a view of the side of a portion of the guide drawn in FIG. 26C. The dotted area represents the fins that extend into the grooves of the first vertebrae.

FIG. 26E is a view of the top of a third guide used to complete the grooves in the second vertebra. The inferior surface of the guide has raised areas that fit into the grooves created in the front half of the vertebra. The slots are used to guide the drill while forming grooves in the back half of the vertebra endplate. FIG. 26F is a view of the side of a portion of the guide drawn in FIG. 26E. The dotted area represents the fins that extend into the front half of the second vertebra endplate.

FIG. 27A is a sagittal cross section of another embodiment of the ADR endplates with an alternative locking mechanism. FIG. 27B is a sagittal cross section of the modular cushioning component that fits into the embodiment of the ADR endplates drawn in FIG. 27A. FIG. 27C is a sagittal cross section of an assembled embodiment of the ADR drawn in FIGS. 27A and 27B. Spring-like projections 2204 from the front of the cushioning component and projections from the rear of the cushioning component snap into spaces in the ADR endplates. FIG. 28 is the view of the side of an alternative embodiment of the ADR. Physical features associated with the top and bottom endplates interact to limit the amount of torsional rotation allowed by the ADR.

The hydrogel embodiments may also use fluid-permeable channels through the ADR endplates and/or the elastomer ring. In addition, although certain of the drawings show the elastomer and the elastomer side of the ADR endplates as flat, they may alternatively be concave or convex. For example, the elastomer ring could have a convex top and bottom and the ADR endplates could have a concave surface against the elastomer ring. Furthermore, an air-filled bag may be used as opposed to a fluid-filled bag. For example, an air-filled bag surrounded by polyurethane or other suitable material may be constructed to function similar to air-cushioned athletic (i.e., Nike) shoes.

We claim:

1. Cushioning apparatus for a joint or artificial disc replacement, comprising:
a central compressible member; and
a component surrounding the central compressible member which expands to dampen transmitted forces.

2. The cushioning apparatus of claim 1, wherein the central compressible member contains saline, biocompatible oils, or other fluids, gasses, gels or polymers.

3. The cushioning apparatus of claim 1, wherein the component surrounding the central compressible member is an elastomer.

4. The cushioning apparatus of claim 1, wherein the component surrounding the central compressible member is an elastomeric ring.

5. The cushioning apparatus of claim 1, wherein the component surrounding the central compressible member includes a hydrogel.

6. The cushioning apparatus of claim 1, further including one or more opposing endplates, each with an endplate surface facing the central compressible member forming an artificial disc replacement.

7. The cushioning apparatus of claim 6, wherein one or both of the endplate surfaces are concave.

8. The cushioning apparatus of claim 6, wherein:
the component surrounding the central compressible member includes a hydrogel; and
one or both of the endplates contain pores for the movement of fluids into and out of the hydrogel.

9. The cushioning apparatus of claim 6, wherein one or both of the endplates include bone-penetrating spikes or projections.

10. The cushioning apparatus of claim 9, wherein the bone-penetrating spikes or projections vary in height.

11. The cushioning apparatus of claim 9, wherein the bone-penetrating spikes or projections closest to the anterior portion of the ADR are longer or shorter than the spikes at the posterior portion of the ADR.

12. The cushioning apparatus of claim 6, wherein one or both of the endplates vary in thickness.

13. The cushioning apparatus of claim 6, including an upper endplate that is thicker than a lower endplate.

14. The cushioning apparatus of claim 6, wherein one or both of the endplates includes a convex bone-contacting surface.

15. The cushioning apparatus of claim 11, wherein the convex bone-contacting surface is cementless.

16. The cushioning apparatus of claim 6, wherein the thickest portion of one or both of the endplates in and ADR application is posterior to the midline.

17. The cushioning apparatus of claim 6, further including a flexible, impermeable membrane sealed to the endplates.

18. The cushioning apparatus of claim 6, further including a component that attaches to one or both of the endplates to prevent the extrusion of material from between the endplates.
